(19) United States
(12) Patent Application Publication
(10) Pub. No.: US 2026/0027253 A1
(43) Pub. Date: Jan. 29, 2026

(54) OIL-LEAKAGE PREVENTION ATOMIZER AND ESSENTIAL OIL DIFFUSER

(71) Applicant: Shenzhen Parlex Household Industrial Co., Ltd., Shenzhen (CN)

(72) Inventor: SikMan TSE, Shenzhen (CN)

(21) Appl. No.: 19/007,529

(22) Filed: Jan. 1, 2025

(30) Foreign Application Priority Data
Jul. 26, 2024 (CN) 202421741239.X

(51) Int. Cl. A61L 9/14

(52) U.S. Cl. CPC A61L 9/14; A61L 2209/133; A61L 2209/134

(57) ABSTRACT

Provided are a leak-proof atomizer and an aroma diffuser. The leak-proof atomizer includes a liquid storage bottle, an end base, and a guide head. The end base is connected with the liquid storage bottle, and is provided with an air cavity with an air inlet and a mist outlet. The guide head is mounted on the end base and is provided with an air inlet hole connected with the air cavity, the air cavity is further provided with an oil outlet hole corresponding to the air inlet, the air inlet hole and an air outlet hole respectively extend into the liquid storage bottle through an outer tube and an inner tube, and the outer tube is sleeved with the inner tube. The aroma diffuser is used for installing the leak-proof atomizer and providing a high-speed air source to the air inlet.

### BACKGROUND OF THE INVENTION

### Field of the Invention

The present invention belongs to the technical field of atomizing aromatherapy equipment, and in particular, relates to a leak-proof atomizer and an aroma diffuser.

### Description of Related Art

The aroma diffuser is a fragrance diffusion product that can transform an essential oil into a fragrant mist for purifying indoor air. Most traditional aroma diffusers are of integrated design, when different types of essential oils are replaced, various essential oils are easily mixed in an essential oil cavity, resulting in a messy smell of the volatile fragrant mist.

Therefore, split-type aroma diffusers appear on the market, the split-type aroma diffuser mainly includes a machine body and a plurality of atomizers filled with different types of essential oils, the plurality of atomizers are detachably connected with the machine body, and various essential oils can be prevented from being mixed by replacing different atomizers, which has a good user experience. However, it was found during use that the existing split-type aroma diffuser has at least the following problems.

First, an atomization chip must be installed at a mist outlet of the atomizer to heat and volatilize the essential oil, resulting in high production cost.

Secondly, when the atomizer is replaced, the atomizer may be inverted, causing the essential oil in the atomizer to overflow from the mist outlet under the action of gravity and contaminate the surrounding area, resulting in a poor user experience.

### SUMMARY OF THE INVENTION

The present invention provides a leak-proof atomizer and an aroma diffuser, which can solve the problems in the background art.

In order to achieve the above-described object, the present invention provides the following technical solutions.

A leak-proof atomizer including: a liquid storage bottle; an end base; and a guide head, in which the end base is connected with a bottle opening portion of the liquid storage bottle and has an air cavity inside, the air cavity is provided with an air inlet and a mist outlet that are connected with the outside on a side wall thereof, and the air inlet is capable of being connected with a high-speed air source, the guide head is mounted on the end base, is located at the bottle opening portion, and is provided with an air inlet hole connected with the air cavity and extending to an inner bottom side of the liquid storage bottle through an outer tube, the air cavity is further provided with an oil outlet hole inside corresponding to the air inlet, and the oil outlet hole penetrates the guide head through an inner tube and extends to the inner bottom side of the liquid storage bottle, and the outer tube is sleeved with the inner tube, and a tube gap is provided between the outer tube and the inner tube.

Preferably, the oil outlet hole is arranged on the guide head.

Preferably, the end base is provided with a guide block inside, and the air inlet and the oil outlet hole are both arranged on the guide block.

Preferably, the leak-proof atomizer further includes a one-way valve that is mounted on the guide block and located in the air inlet, and has an end facing the oil outlet hole.

Preferably, the end base is provided with a sleeve, the guide head is located in the sleeve, the bottle opening portion of the liquid storage bottle is threadably connected to the sleeve and abuts against the guide head, and a bottom end of the guide head penetrates the bottle opening portion into the liquid storage bottle.

Preferably, the leak-proof atomizer further includes a tube valve that is located in the liquid storage bottle and is mounted on an end of the outer tube away from the guide head, the inner tube extends into the tube valve, the tube valve is further provided with a valve hole, and both the outer tube and the inner tube are connected with the liquid storage bottle through the valve hole.

Preferably, the air inlet is designed in a conical shape and gradually shrinks in a direction approaching the air cavity, and the oil outlet hole is designed in a conical shape and gradually shrinks in the direction approaching the air cavity.

Preferably, the leak-proof atomizer further includes a partition shell that is mounted in the end base, isolates the mist outlet from the air cavity, and is provided with a plurality of diffusion ports, the plurality of diffusion ports are staggered with the oil outlet hole and are connected with the air cavity and the mist outlet.

Preferably, the partition shell is further provided with a guide column coaxially arranged below the mist outlet.

An aroma diffuser including: a machine body; an air supply unit; and the leak-proof atomizer, in which the leak-proof atomizer is detachably mounted on the machine body, the air supply unit is mounted inside the machine body and is correspondingly connected with the air inlet of the end base to continuously deliver a high-speed airflow to the oil outlet hole to generate a negative pressure, thereby extracting an essential oil in the liquid storage bottle and spraying the essential oil to the outside from the mist outlet.

Preferably, the machine body has an accommodating cavity inside, a top portion of the accommodating cavity is provided with a positioning slot hole connected with the outside, the liquid storage bottle is allowed to enter and exit the accommodating cavity through the positioning slot hole, and the end base is capable of being inserted into or separated from the positioning slot hole, so that the leak-proof atomizer is detachably connected with the machine body, and when the end base is inserted into the positioning slot hole, the liquid storage bottle is located in the accommodating cavity, and the air inlet is connected with the air supply unit.

Preferably, the air supply unit includes a sleeve housing and an air pump, the sleeve housing is mounted inside the accommodating cavity and is capable of being coaxially sleeved with the end base, the sleeve housing has an air duct inside, and the air pump is mounted inside the accommodating cavity and is connected with the air duct, and when the end base is inserted into the positioning slot hole, the air inlet is aligned with and connected with the air duct.

Preferably, the end base is rotatably matched with the positioning slot hole, the end base is provided with a block, and the sleeve housing is further provided with an annular air groove connected with the air duct, and when the end base is inserted into the positioning slot hole, the block abuts against a top portion of the machine body, and the air inlet is connected with any part of the annular air groove.

Preferably, a cover shell is detachably mounted on the top portion of the machine body, the cover shell is used for covering or opening the leak-proof atomizer, and the cover shell is provided with a through opening correspondingly connected with the mist outlet.

The present invention provides the leak-proof atomizer and the aroma diffuser. The leak-proof atomizer is designed with the air inlet to continuously supply the high-speed airflow to the oil outlet hole to form the negative pressure, so as to smoothly extract the essential oil in the liquid storage bottle into the air cavity for diffusion, and spray the diffused essential oil to the outside from the mist outlet, and by designing the inner tube and the outer tube on the guide head, the gas in the air cavity is guided to the liquid storage bottle and the essential oil in the liquid storage bottle is guided to the air cavity at the same time, so that air pressures inside and outside the liquid storage bottle are balanced to maintain normal oil supply. Compared with the prior art, this structural design is not only simple in structure, but also does not require an additional design of an atomization chip, which can effectively reduce the production cost. Both the outer tube and the inner tube extend to the inner bottom side of the liquid storage bottle, and thus when the liquid storage bottle is inverted, both the outer tube and the inner tube are exposed to an essential oil liquid level to prevent the essential oil from leaking from the air inlet hole and the oil outlet hole and contaminating the surrounding area, which effectively improves the user experience. Due to the unique design of the leak-proof atomizer, the aroma diffuser equipped with the leak-proof atomizer does not cause the essential oil to spill when the atomizer is replaced and attached, thereby improving the user experience. Furthermore, the machine body and the leak-proof atomizer are independent of each other, and thus when the leak-proof atomizer or the machine body is damaged, they can be repaired separately, thereby reducing the maintenance cost.

The accompanying drawings are used to provide a further understanding of the present invention and constitute a part of the specification, and the accompanying drawings together with the embodiments of the present invention are used to describe the present invention and do not limit the present invention.

### BRIEF DESCRIPTION OF THE DRAWINGS

FIG. 1 shows a schematic diagram of an overall structure of the present invention.

FIG. 2 shows a cross-sectional view of FIG. 1.

FIG. 3 shows an enlarged view of part A in FIG. 2.

FIG. 4 shows an exploded schematic diagram of FIG. 1.

FIG. 5 shows a schematic structural diagram of a machine body according to the present invention.

FIG. 6 shows a cross-sectional view of FIG. 5.

FIG. 7 shows an enlarged view of part B in FIG. 6.

FIG. 8 shows a schematic structural diagram of a leak-proof atomizer according to a first embodiment of the present invention.

FIG. 9 shows a cross-sectional view of FIG. 8.

FIG. 10 shows an enlarged view of part C in FIG. 9.

FIG. 11 shows an exploded schematic diagram of FIG. 8.

FIG. 12 shows an enlarged view of part D in FIG. 11.

FIG. 13 shows a first exploded schematic diagram of an end base of the present invention.

FIG. 14 shows a second exploded schematic diagram of the end base of the present invention.

FIG. 15 shows a schematic structural diagram of a guide head in FIG. 11.

FIG. 16 shows a cross-sectional view of FIG. 15.

FIG. 17 shows a schematic structural diagram of a leak-proof atomizer according to a second embodiment of the present invention.

FIG. 18 shows a cross-sectional view of FIG. 17.

FIG. 19 shows an enlarged view of part E in FIG. 18.

FIG. 20 shows a schematic structural diagram of the guide head in FIG. 18.

### DETAILED DESCRIPTION OF THE INVENTION

The technical solutions in the embodiments of the present application will be clearly and completely described below with reference to the accompanying drawings in the embodiments of the present application. Obviously, the described embodiments are merely part of the embodiments of the present application, not all of the embodiments. Based on the embodiments in the present application, all other embodiments obtained by those skilled in the art without creative work fall within the protection scope of the present application. The accompanying drawings are merely provided for reference and description and are not intended to limit the present application. A connection relation shown in the accompanying drawings is merely for the convenience of clear description, and does not limit a connection manner.

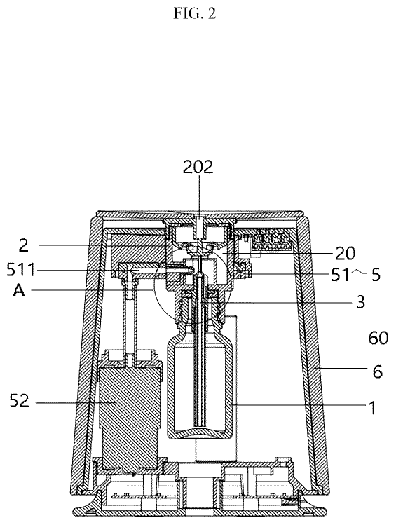

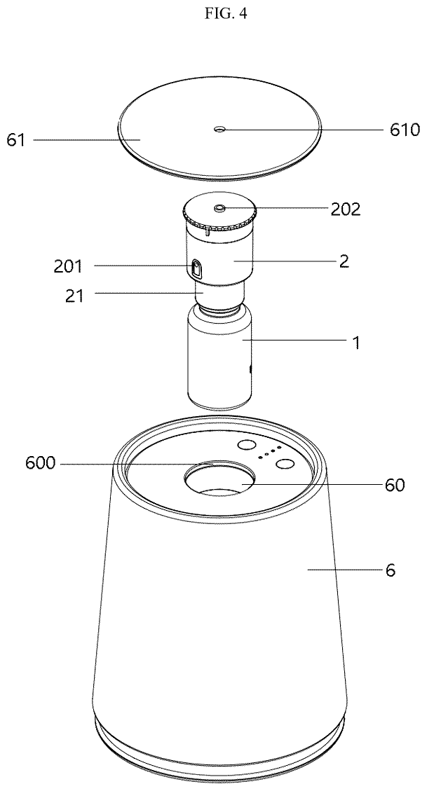

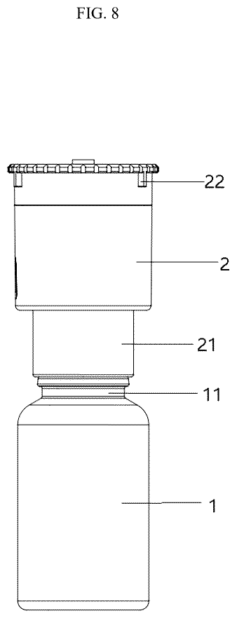

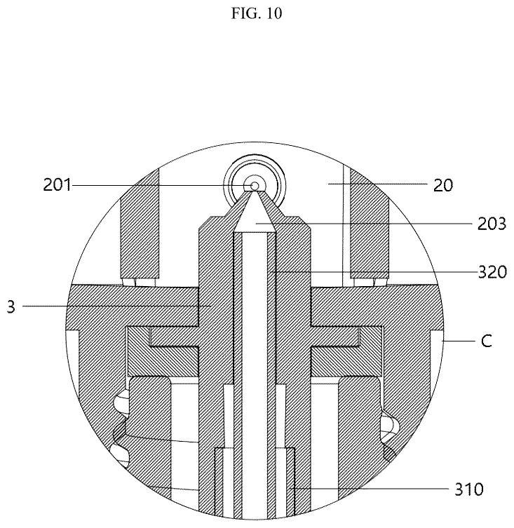

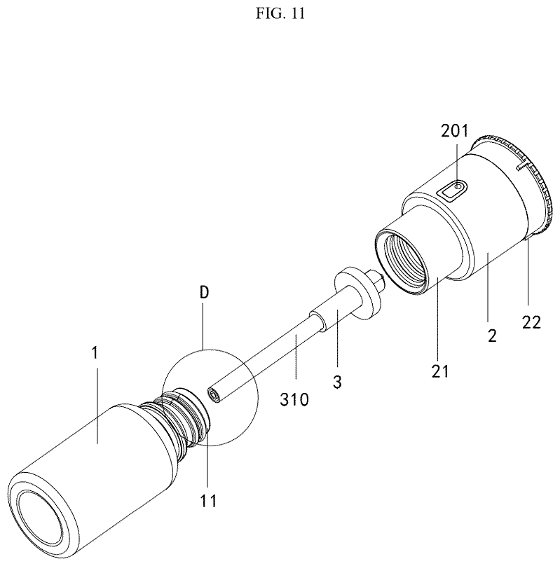

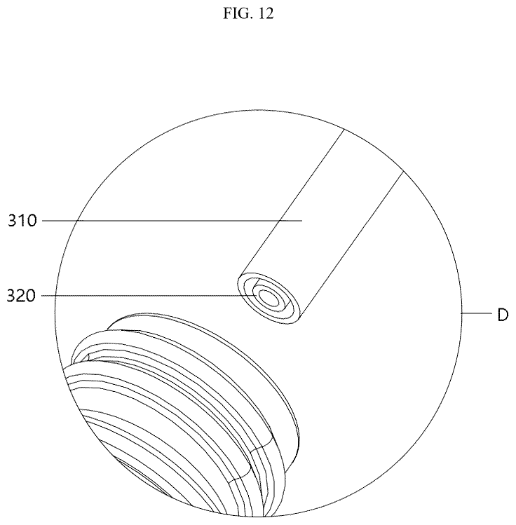

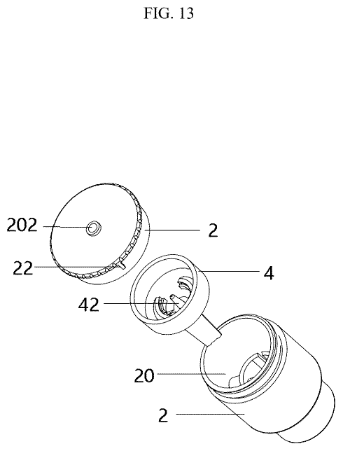

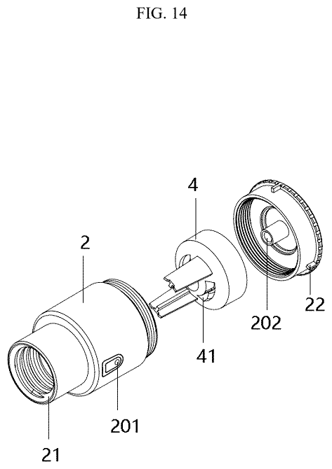

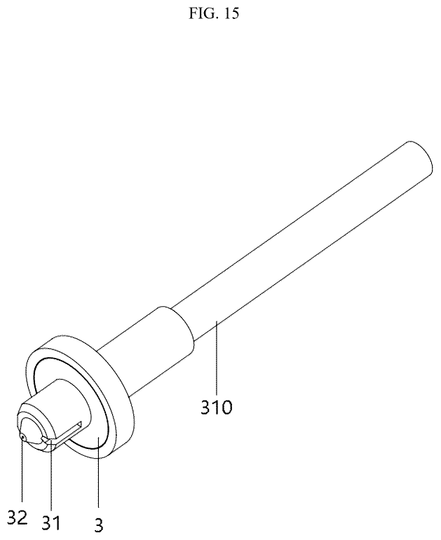

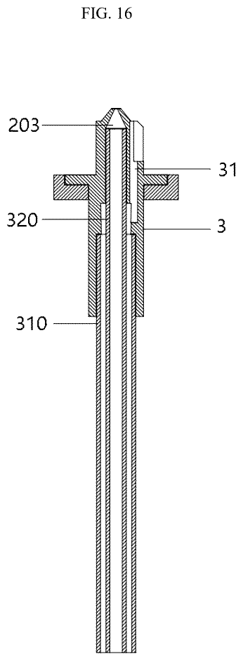

Referring to FIGS. 2 to 4 and 8 to 16, a leak-proof atomizer includes a liquid storage bottle 1, an end base 2, and a guide head 3. The end base 2 is connected with a bottle opening portion 11 of the liquid storage bottle 1 and has an air cavity 20 inside, the air cavity 20 is provided with an air inlet 201 and a mist outlet 202 that are connected with the outside on a side wall thereof, and the air inlet 201 is capable of being connected with a high-speed air source. The guide head 3 is mounted on the end base 2, is located at the bottle opening portion 11, and is provided with an air inlet hole 31 connected with the air cavity 20, the air inlet hole 31 extends to an inner bottom side of the liquid storage bottle 1 through an outer tube 310, the air cavity 20 is further provided with an oil outlet hole 203 inside corresponding to the air inlet 201, the oil outlet hole 203 penetrates the guide head 3 through an inner tube 320 and extends to the inner bottom side of the liquid storage bottle 1, the outer tube 310 is sleeved with the inner tube 320, and a tube gap 30 is provided between the outer tube 310 and the inner tube 320.

Specifically, during use, the air inlet 201 is connected with the high-speed air source, and a high-speed airflow enters the air cavity to continuously carry away gas at the oil outlet hole 203, and discharge the gas to the outside through the mist outlet 202, thereby forming a negative pressure in the inner tube 320 to pump an essential oil in the liquid storage bottle 1 into the air cavity 20. The essential oil entering the air cylinder 20 is brought into contact with the high-speed airflow and diffuses, and finally follows the high-speed airflow to be ejected from the mist outlet 202 to the outside to form a fragrant mist. While the inner tube 320 is pumping the essential oil in the liquid storage bottle 1, the gas in the air cavity 20 simultaneously enters the liquid storage bottle 1 through the air inlet hole 31, thereby maintaining a balance of air pressures inside and outside the liquid storage bottle 1 and ensuring that the essential oil in the liquid storage bottle 1 can flow out normally. In addition, the air inlet hole 31 is connected with the liquid storage bottle 1, and thus the essential oil that is not ejected to the outside and remains in the air cavity 20 flows back into the liquid storage bottle 1 through the air inlet hole 31, thereby avoiding waste. In the above-described operation process, no atomization chip is needed, which saves the cost.

When the leak-proof atomizer is replaced, if the liquid storage bottle 1 is inverted, both the outer tube 310 and the inner tube 320 are exposed to an essential oil liquid level to prevent the essential oil in the liquid storage bottle 1 from continuously flowing into the air cavity 20 from the air inlet hole 31 and the oil outlet hole 203 through the outer tube 310 and the inner tube 320, thereby solving a problem that the essential oil is easy to overflow and leak when the liquid storage bottle 1 is inverted. Further, even if the essential oil partially remaining in the outer tube 310 and the inner tube 320 flows into the air cavity 20 under the action of gravity, the essential oil in the air cavity 20 can re-flow back into the liquid storage bottle 1 through the air inlet hole 31 when a user places the liquid storage bottle 1 upright, thereby effectively improving the user experience.

The design of the outer tube 310 sleeved with the inner tube 320 can optimize an installation space of the outer tube 310 and the inner tube 320, and can effectively reduce a volume of the guide head 3 and can prevent the outer tube 310 and the inner tube 320 from squeezing the bottle opening portion 11 of the liquid storage bottle 1 due to a large space occupied by the outer tube 310 and the inner tube 320, thereby improving the installation convenience. The tube gap 30 is provided between the outer tube 310 and the inner tube 320, so that the essential oil or the gas can flow normally between the air cavity 20 and the liquid storage bottle 1, and during the inversion of the liquid storage bottle 1, under the action of tension of the liquid, a water film can be quickly formed at a connecting port 300 between the tube gap 30 and the liquid storage bottle 1 to prevent the essential oil in the liquid storage bottle 1 from continuously flowing into the outer tube 310 and the inner tube 320 at a moment of inverting the liquid storage bottle 1, resulting in an excessive essential oil entering the air cavity 20 and overflowing.

In summary, the present invention forms a negative pressure by designing the air inlet 201 to continuously supply a high-speed airflow to the oil outlet hole 203, so as to smoothly extract the essential oil in the liquid storage bottle 1 into the air cavity 20 for diffusion, and spray the diffused essential oil to the outside from the mist outlet 202. By designing the inner tube 320 and the outer tube 310 on the guide head 3, the gas in the air cavity 20 is guided to the liquid storage bottle 1 and the essential oil in the liquid storage bottle 1 is guided to the air cavity 20 at the same time, so that the air pressures inside and outside the liquid storage bottle 1 are balanced to maintain normal oil supply. Compared with the prior art, this structural design is not only simple in structure, but also does not require an additional design of an atomization chip, which can effectively reduce the production cost. Both the outer tube 310 and the inner tube 320 extend to the inner bottom side of the liquid storage bottle 1, and thus when the liquid storage bottle 1 is inverted, both the outer tube 310 and the inner tube 320 are exposed to the essential oil liquid level to prevent the essential oil from continuously leaking from the air inlet hole 31 and the oil outlet hole 203 and contaminating the surrounding area, which effectively improves the user experience.

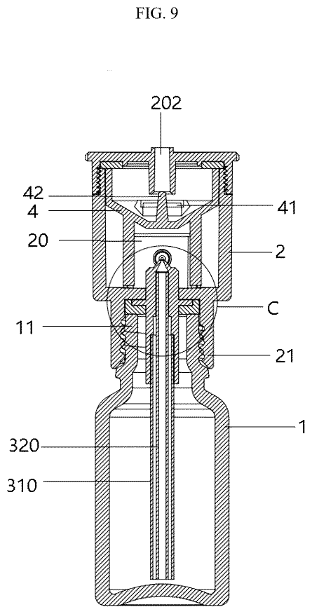

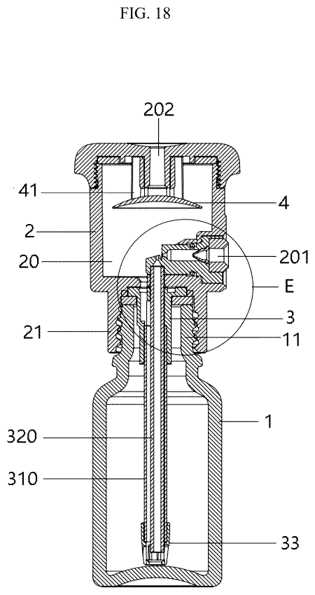

Further, the outer tube 310 and the inner tube 320 may adopt a coaxial design as shown in FIG. 9, or may adopt an offset design as shown in FIG. 18, which is not limited in the present invention.

Furthermore, regarding a position design of the oil outlet hole 203, there are at least two embodiments.

In a first embodiment, as shown in FIGS. 2, 3, and 8 to 11, the oil outlet hole 203 is directly arranged on the guide head 3. Under this design, the installation of the inner tube 320 is facilitated, the assembly convenience is high, but the actual atomization effect is poor.

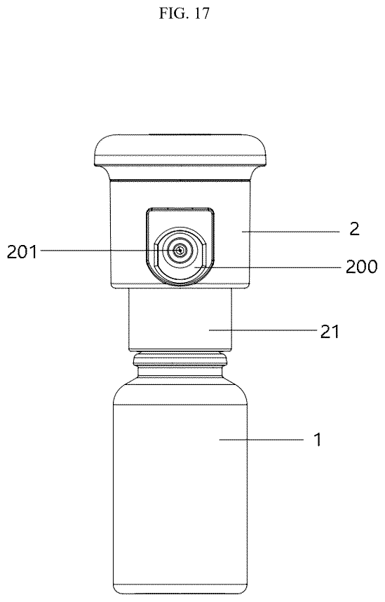

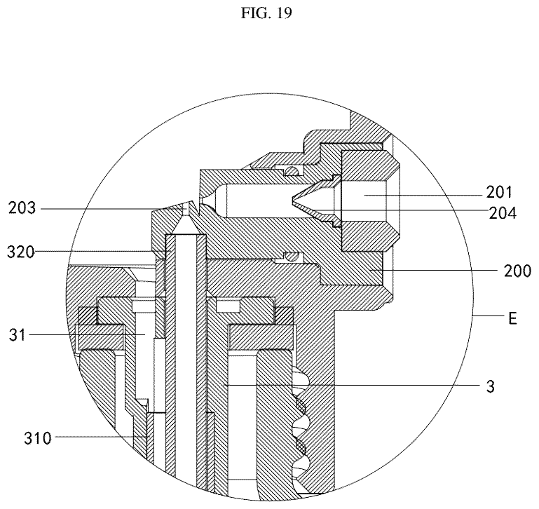

In a second embodiment, as shown in FIGS. 17 to 20, the end base 2 is provided with a guide block 200 inside, and the air inlet 201 and the oil outlet hole 203 are both arranged on the guide block 200. Under this design, a high-pressure airflow input from the air inlet 201 can more easily impact the oil outlet hole 203 to form a negative pressure, which has a better atomization effect.

In an actual production process, corresponding designs may be adopted according to the requirements, which are not limited in the present invention.

Referring to FIGS. 17 to 20, the present invention further includes a one-way valve 204 that is mounted on the guide block 200 and located in the air inlet 201, and has an end facing the oil outlet hole 203. This design enables the airflow to enter the air cavity 20 only through the air inlet 201, thereby preventing an atomized essential oil gas in the air cavity 20 from flowing back and overflowing through the air inlet 201.

Referring to FIGS. 9 to 18, the end base 2 is provided with a sleeve 21, the guide head 3 is located in the sleeve 21, the bottle opening portion 11 of the liquid storage bottle 1 is threadably connected to the sleeve 21 and abuts against the guide head 3, and a bottom end of the guide head 3 penetrates the bottle opening portion 11 into the liquid storage bottle 1.

Specifically, during installation, the guide head 3 is placed in the sleeve 21 and then the liquid storage bottle 1 is tightened, so that the bottle opening portion 11 of the liquid storage bottle 1 abuts against the guide head 3. At this time, the bottom end of the guide head 3 penetrates the bottle opening portion 11 into the liquid storage bottle 1, and the air cavity 20 and the liquid storage bottle 1 are connected through the air inlet hole 31, the oil outlet hole 203, the outer tube 310, and the inner tube 320. The above-described design facilitates the assembly between the end base 2, the guide head 3, and the liquid storage bottle 1, and improves the practicality, and since the liquid storage bottle 1 and the end base 2 are detachably connected to each other, the user can adjust a smell by directly replacing the liquid storage bottle 1 containing different essential oils, which further reduces the cost of use.

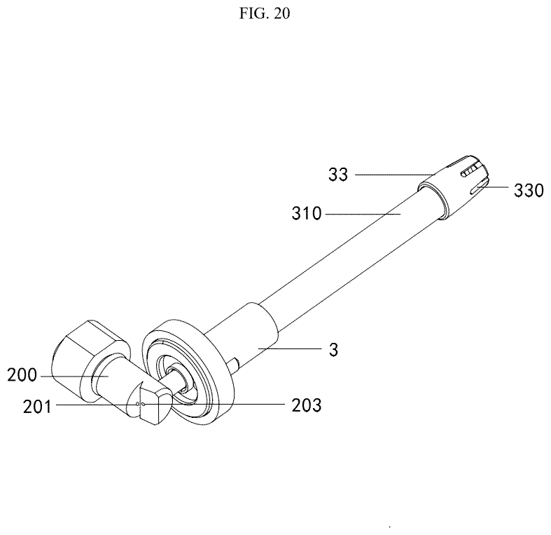

Referring to FIGS. 18 and 20, the present invention further includes a tube valve 33 that is located in the liquid storage bottle 1 and is mounted on an end of the outer tube 310 away from the guide head 3, the inner tube 320 extends into the tube valve 33, the tube valve 33 is further provided with a valve hole 330, and both the outer tube 310 and the inner tube 320 are connected with the liquid storage bottle 1 through the valve hole 330.

Specifically, during use, the gas in the air cavity 20 and the essential oil in the liquid storage bottle 1 both pass through the valve hole 330, and the valve hole 330 can slow down a flow rate of the gas and a flow rate of the essential oil to prevent the essential oil from being lost too quickly. On the other hand, under the action of tension of the liquid, a thin film may be formed at the valve hole 330 to prevent the essential oil in the liquid storage bottle 1 from entering the outer tube 310 and the inner tube 320 at the moment of inverting the liquid storage bottle 1.

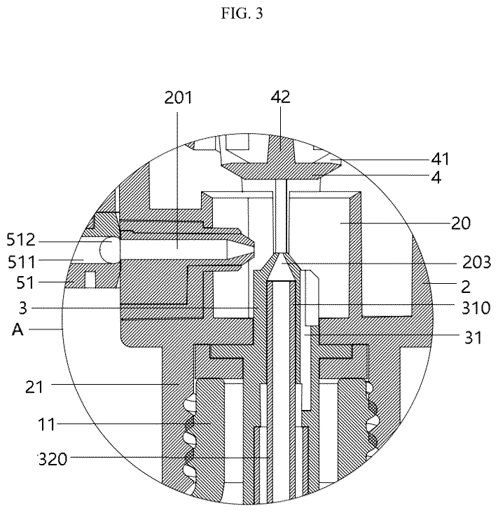

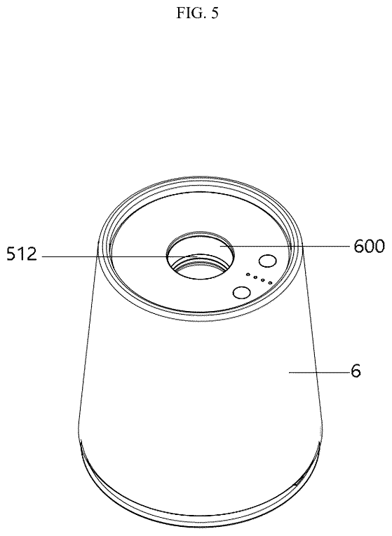

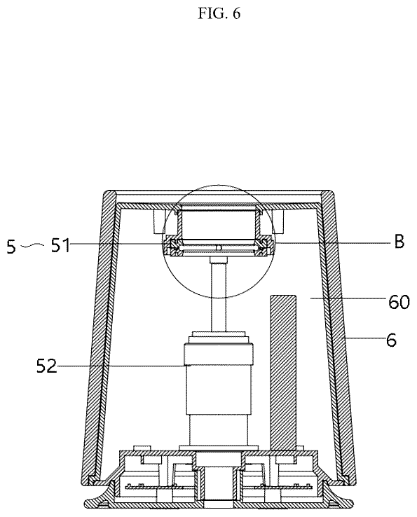

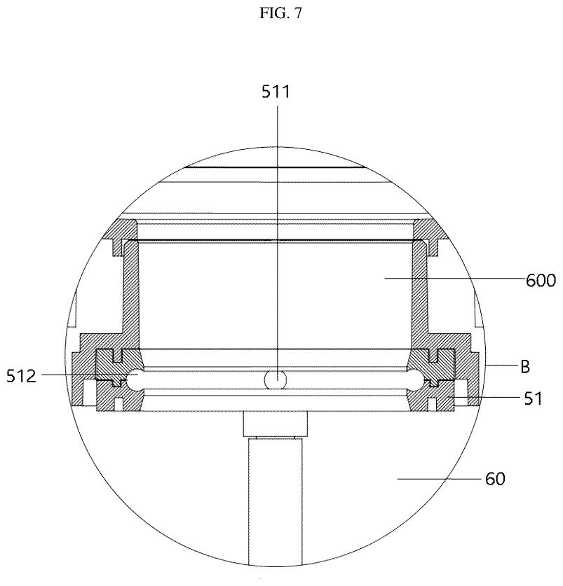

Referring to FIGS. 3 to 10, the air inlet 201 is designed in a conical shape and gradually shrinks in a direction approaching the air cavity 20, and this design enables the high-speed airflow entering the air cavity 20 to be concentrated and increase a wind force, thereby ensuring that the high-speed airflow can smoothly carry away the gas at the oil outlet hole 203 and strongly disperse the essential oil entering the air cavity 20. The oil outlet hole 203 is designed in a conical shape and gradually shrinks in the direction approaching the air cavity 20, and this design can accelerate the generation of the negative pressure in the inner tube 320, and can reduce a discharge amount of essential oil, thereby ensuring that the high-speed airflow in the air cavity 20 can fully be brought into contact with the essential oil, thereby accelerating a speed at which the essential oil is blown into a mist. The two are used in combination, and thus the essential oil in the liquid storage bottle 1 can be stably pumped into the air cavity 20 and formed into mist.

Referring to FIGS. 3, 13, and 14, the present invention further includes a partition shell 4 that is mounted in the end base 2, isolates the mist outlet 202 from the air cavity 20, and is provided with a plurality of diffusion ports 41, and the plurality of diffusion ports 41 are staggered with the oil outlet hole 203 and are connected with the air cavity 20 and the mist outlet 202.

Specifically, the mist essential oil in the air cavity 20 moves toward the plurality of diffusion ports 41 driven by the high-speed airflow, and is finally sprayed to the outside through the mist outlet 202. Therefore, the design of the partition shell 4 and the plurality of diffusion ports 41 can guide and disperse the mist essential oil, and effectively reduce a possibility of the mist essential oil being remixed in the air cavity 20 and causing residue, thereby improving a utilization rate of the essential oil and the aromatherapy effect.

Referring to FIGS. 3, 13, and 14, the partition shell 4 is further provided with a guide column 42 that is coaxially arranged below the mist outlet 202 and guides the mist essential oil to move toward the mist outlet 202, thereby reducing a possibility of the mist essential oil flowing back into the air cavity 20.

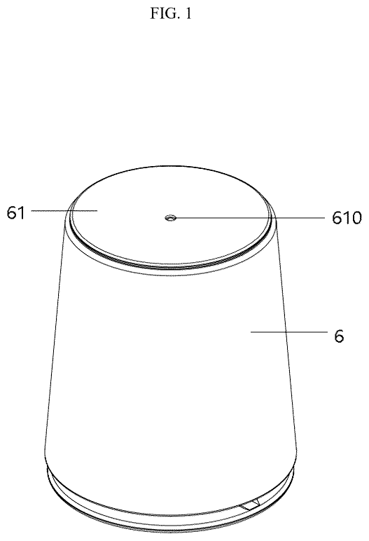

Referring to FIGS. 1 to 7, an aroma diffuser includes a machine body 6, an air supply unit 5, and the leak-proof atomizer. The leak-proof atomizer is detachably mounted on the machine body 6, and the air supply unit 5 is mounted inside the machine body 6 and is correspondingly connected with the air inlet 201 of the end base 2 to continuously deliver the high-speed airflow to the oil outlet hole 203 to generate a negative pressure, thereby extracting the essential oil in the liquid storage bottle 1 and spraying the essential oil to the outside from the mist outlet 202.

Specifically, during use, according to the requirements, a leak-proof atomizer containing a corresponding scent essential oil is selected and mounted on the machine body 6, and then the air supply unit 5 is started. The high-speed airflow supplied by the air supply unit 5 enters the air cavity 20 through the air inlet 201, and continuously drives the air at the oil outlet hole 203 to generate a negative pressure in the inner tube 320. At this time, the essential oil in the liquid storage bottle 1 moves along the inner tube 320 to the air cavity 20 under the action of air pressure, and is brought into contact with the high-speed airflow in the air cavity 20 to be dispersed into mist, and the mist essential oil is finally discharged from the mist outlet 202 by the high-speed airflow and forms an aromatherapy spray in the outside. After the essential oil is used up, the air supply unit 5 is closed, the leak-proof atomizer is detached from the machine body 6, and then a new leak-proof atomizer is replaced.

In summary, due to the unique design of the leak-proof atomizer, the aroma diffuser equipped with the leak-proof atomizer does not cause the essential oil to spill even if the atomizer is inverted when being replaced and attached, thereby improving the user experience. Furthermore, the machine body 6 and the leak-proof atomizer are independent of each other, and thus when the leak-proof atomizer or the machine body 6 is damaged, they can be repaired separately, thereby reducing the maintenance cost.

Referring to FIGS. 1 to 7, there are various manners of assembling the machine body 6 and the leak-proof atomizer, which are not limited in the present invention. For ease of understanding, in the present embodiment, the machine body 6 has an accommodating cavity 60 inside, a top portion of the accommodating cavity 60 is provided with a positioning slot hole 600 connected with the outside, the liquid storage bottle 1 is allowed to enter and exit the accommodating cavity 60 through the positioning slot hole 600, and the end base 2 is capable of being inserted into or separated from the positioning slot hole 600, so that the leak-proof atomizer is detachably connected with the machine body 6, and when the end base 2 is inserted into the positioning slot hole 600, the liquid storage bottle 1 is located in the accommodating cavity 60, and the air inlet 201 is connected with the air supply unit 5.

Specifically, when the leak-proof atomizer is attached, the end base 2 is held by hand and the liquid storage bottle 1 is aligned with the positioning slot hole 600, then the end base 2 is moved downward so that the liquid storage bottle 1 passes through the positioning slot hole 600 into the accommodating cavity 60. At this time, the end base 2 is inserted into the positioning slot hole 600, and the air inlet 201 is connected with the air supply unit 5, so that the leak-proof atomizer can be sprayed normally by starting the air supply unit 5. The accommodating cavity 60 may be designed to hide the liquid storage bottle 1, so as to reduce the space occupied and improve the overall aesthetics.

Referring to FIGS. 1 to 7, the air supply unit 5 includes a sleeve housing 51 and an air pump 52, the sleeve housing 51 is mounted inside the accommodating cavity 60 and is capable of being coaxially sleeved with the end base 2, the sleeve housing 51 has an air duct 511 inside, which can be aligned with the air inlet 201, and the air pump 52 is mounted inside the accommodating cavity 60 and is connected with the air duct 511.

Specifically, when the end base 2 is inserted into the positioning slot hole 600, the sleeve housing 51 is coaxially sleeved with the end base 2, and the air inlet 201 is aligned with and is connected with the air duct 511. At this time, the air pump 52 is started to supply a high-speed airflow to the air duct 511, so that the high-speed airflow can enter the air cavity 20 through the air inlet 201, and continuously carry away the gas at the oil outlet hole 203 to generate a negative pressure.

The design of the sleeve housing 51 may provide support for the end base 2, so as to further improve the installation stability of the end base 2, and ensure that the air duct 511 can tightly be connected with the air inlet 201, thereby improving the air supply stability. In addition, the air pump 52 is an existing product, and thus a specific structure and working principle thereof are not further described in the present invention. Further, the sleeve housing 51 is provided with a sealing ring (not shown) in contact with an outer side wall of the end base 2 at an inner side thereof, so as to increase the sealing performance after the sleeve housing 51 is matched with the end base 2, thereby avoiding air leakage between the air duct 511 and the air inlet 201.

Referring to FIGS. 1 to 7, the end base 2 is rotatably matched with the positioning slot hole 600, the end base 2 is provided with a block 22, and the sleeve housing 51 is further provided with an annular air groove 512 connected with the air duct 511, and when the end base 2 is inserted into the positioning slot hole 600, the block 22 abuts against a top portion of the machine body 6, and the air inlet 201 is connected with any part of the annular air groove 512.

Specifically, the end base 2 and the positioning slot hole 600 are designed to be rotatably matched with each other, when the user attaches the leak-proof atomizer, the user can complete the assembly of the leak-proof atomizer by directly inserting the end base 2 into the positioning slot hole 600 and enabling the block 22 to abut against the top portion of the machine body 6 without adjusting an angle of the end base 2. With the matching design of the annular air groove 512 and the air inlet 201, when the end base 2 is inserted into the positioning slot hole 600, the air inlet 201 can be correspondingly connected with any part of the annular air groove 512, thereby ensuring that the air inlet 201 can be connected with the air duct 511 in all directions. Therefore, the above-described design can improve the installation convenience of the leak-proof atomizer, shorten the installation time, and can avoid the air inlet 201 from staggering the air duct 511, thereby improving the installation convenience and the user experience.

Referring to FIGS. 1 to 7, a cover shell 61 is detachably mounted on the top portion of the machine body 6, is used for covering or opening the leak-proof atomizer, and is provided with a through opening 610 correspondingly connected with the mist outlet 202.

Specifically, when the cover shell 61 covers the leak-proof atomizer, the cover shell 61 can abut against the end base 2 to prevent the end base 2 from moving upward and separating from the positioning slot hole 600, and the through opening 610 is used for normal mist discharge. The cover shell 61 can be fixed to the machine body 6 by a method such as snap connection or magnetic connection, which is not limited in the present invention.

Although the embodiments of the present application have been shown and described, it will be understood by those skilled in the art that various changes, modifications, substitutions, and variations may be made to these embodiments without departing from the principles and spirit of the present application.

### REFERENCE SIGNS LIST

1: liquid storage bottle
11: bottle opening
portion 2: end base
20: air cavity
201: air inlet
202: mist outlet
203: oil outlet hole
204: one-way valve
21: sleeve
22: block
3: guide head
30: tube gap
300: connecting port
31: air inlet hole
310: outer tube
320: inner tube
33: tube valve
330: valve hole
4: partition shell
41: diffusion port
42: guide column
5: air supply unit
51: sleeve housing
511: air duct
512: annular air groove
52: air pump
6: machine body
60: accommodating cavity
600: positioning slot hole
61: cover shell
610: through opening

What is claimed is:

1. A leak-proof atomizer
comprising: a liquid storage bottle;
an end base that is connected with a bottle opening portion of the liquid storage bottle and has an air cavity inside, the air cavity being provided with an air inlet and a mist outlet that are connected with the outside on a side wall thereof, and the air inlet capable of being connected with a high-speed air source; and
a guide head that is mounted on the end base, is located at the bottle opening portion, and is provided with an air inlet hole connected with the air cavity and extending to an inner bottom side of the liquid storage bottle through an outer tube, wherein the air cavity is further provided with an oil outlet hole inside corresponding to the air inlet, and the oil outlet hole penetrates the guide head through an inner tube and extends to the inner bottom side of the liquid storage bottle, and the outer tube is sleeved with the inner tube, and a tube gap is provided between the outer tube and the inner tube.

2. The leak-proof atomizer according to claim 1, wherein the oil outlet hole is arranged on the guide head.

3. The leak-proof atomizer according to claim 1, wherein the end base is provided with a guide block inside, and the air inlet and the oil outlet hole are both arranged on the guide block.

4. The leak-proof atomizer according to claim 3, further comprising: a one-way valve that is mounted on the guide block and located in the air inlet, and has an end facing the oil outlet hole.

5. The leak-proof atomizer according to claim 1, wherein the end base is provided with a sleeve, the guide head is located in the sleeve, the bottle opening portion of the liquid storage bottle is threadably connected to the sleeve and abuts against the guide head, and a bottom end of the guide head penetrates the bottle opening portion into the liquid storage bottle.

6. The leak-proof atomizer according to claim 1, further comprising: a partition shell that is mounted in the end base, isolates the mist outlet from the air cavity, and is provided with a plurality of diffusion ports, wherein the plurality of diffusion ports are staggered with the oil outlet hole and are connected with the air cavity and the mist outlet.

7. An aroma diffuser comprising: an air supply unit; a machine body; and the leak-proof atomizer according to claim 1, wherein the leak-proof atomizer is detachably mounted on the machine body, the air supply unit is mounted inside the machine body and is correspondingly connected with the air inlet of the end base to continuously deliver a high-speed airflow to the oil outlet hole to generate a negative pressure, thereby extracting an essential oil in the liquid storage bottle and spraying the essential oil to the outside from the mist outlet.

8. The aroma diffuser according to claim 7, wherein the machine body has an accommodating cavity inside, a top portion of the accommodating cavity is provided with a positioning slot hole connected with the outside, the liquid storage bottle is allowed to enter and exit the accommodating cavity through the positioning slot hole, and
the end base is capable of being inserted into or separated from the positioning slot hole, so that the leak-proof atomizer is detachably connected with the machine body, and when the end base is inserted into the positioning slot hole, the liquid storage bottle is located in the accommodating cavity, and the air inlet is connected with the air supply unit.

9. The aroma diffuser according to claim 8, wherein the air supply unit includes a sleeve housing and an air pump, the sleeve housing is mounted inside the accommodating cavity and is capable of being coaxially sleeved with the end base, the sleeve housing has an air duct inside, and the air pump is mounted inside the accommodating cavity and is connected with the air duct, and when the end base is inserted into the positioning slot hole, the air inlet is aligned with and connected with the air duct.

10. The aroma diffuser according to claim 9, wherein the end base is rotatably matched with the positioning slot hole, the end base is provided with a block, and the sleeve housing is further provided with an annular air groove connected with the air duct, and when the end base is inserted into the positioning slot hole, the block abuts against a top portion of the machine body, and the air inlet is connected with any part of the annular air groove.

11. An aroma diffuser comprising: an air supply unit; a machine body; and the leak-proof atomizer according to claim 2, wherein the leak-proof atomizer is detachably mounted on the machine body, the air supply unit is mounted inside the machine body and is correspondingly connected with the air inlet of the end base to continuously deliver a high-speed airflow to the oil outlet hole to generate a negative pressure, thereby extracting an essential oil in the liquid storage bottle and spraying the essential oil to the outside from the mist outlet.

12. An aroma diffuser comprising: an air supply unit; a machine body; and the leak-proof atomizer according to claim 3, wherein the leak-proof atomizer is detachably mounted on the machine body, the air supply unit is mounted inside the machine body and is correspondingly connected with the air inlet of the end base to continuously deliver a high-speed airflow to the oil outlet hole to generate a negative pressure, thereby extracting an essential oil in the liquid storage bottle and spraying the essential oil to the outside from the mist outlet.

13. An aroma diffuser comprising: an air supply unit; a machine body; and the leak-proof atomizer according to claim 4, wherein the leak-proof atomizer is detachably mounted on the machine body, the air supply unit is mounted inside the machine body and is correspondingly connected with the air inlet of the end base to continuously deliver a high-speed airflow to the oil outlet hole to generate a negative pressure, thereby extracting an essential oil in the liquid storage bottle and spraying the essential oil to the outside from the mist outlet.

14. An aroma diffuser comprising: an air supply unit; a machine body; and the leak-proof atomizer according to claim 5, wherein the leak-proof atomizer is detachably mounted on the machine body, the air supply unit is mounted inside the machine body and is correspondingly connected with the air inlet of the end base to continuously deliver a high-speed airflow to the oil outlet hole to generate a negative pressure, thereby extracting an essential oil in the liquid storage bottle and spraying the essential oil to the outside from the mist outlet.

15. An aroma diffuser comprising: an air supply unit; a machine body; and the leak-proof atomizer according to claim 6, wherein the leak-proof atomizer is detachably mounted on the machine body, the air supply unit is mounted inside the machine body and is correspondingly connected with the air inlet of the end base to continuously deliver a high-speed airflow to the oil outlet hole to generate a negative pressure, thereby extracting an essential oil in the liquid storage bottle and spraying the essential oil to the outside from the mist outlet.
